(19) United States
(12) Patent Application Publication
(10) Pub. No.: US 2026/0357030 A1
(43) Pub. Date: Nov. 12, 2026

(54) MICROWAVE RADIATION MEDIATED DEPOLYMERISATION OF HALOGENATED PLASTICS

(71) Applicant: HALOCYCLE LIMITED, Hull, East Yorkshire (GB)

(72) Inventor: John ROBINSON, Hull, East Yorkshire (GB)

(73) Assignee: HALOCYCLE LIMITED, Hull, East Yorkshire (GB)

(21) Appl. No.: 19/103,084

(22) PCT Filed: Aug. 16, 2023
(86) PCT No.: PCT/GB2023/052157

§ 371 Date: Feb. 11, 2025

(30) Foreign Application Priority Data
Aug. 19, 2022 (GB) 2212121.4

(51) Int. Cl. C08J 11/18

(52) U.S. Cl. CPC C08J 11/18; C08J 2300/102; C08J 2327/06

(57) ABSTRACT

A method of processing a feedstock, wherein the feedstock includes halogenated polymer particles, wherein the halogenated polymer particles have a polymer type that has a first pre-determined solubility parameter. The method includes pre-treating the feedstock by applying a solvent to the feedstock, wherein the solvent has a second pre-determined solubility parameter. The second pre-determined solubility parameter substantially matches the first solubility parameter. The method includes irradiating the feedstock with microwave radiation using a microwave reactor following pre-treating.

### CROSS-REFERENCE TO RELATED APPLICATIONS

This patent application is a U.S. national stage application of, and claims priority to and all benefits of, International Patent Application PCT/GB2023/052157 filed Aug. 16, 2023, which claims priority to GB 2212121.4 filed Aug. 19, 2022, both of which are herein incorporated by reference in their entireties.

### FIELD OF THE DISCLOSURE

The disclosure relates to processing of plastics, and in particular to depolymerisation of halogenated plastics. Aspects of the disclosure relate to processing of a plastics feedstock, and in particular pre-treatment of a plastics feedstock before microwave irradiation in a pyrolysis process.

### BACKGROUND TO THE DISCLOSURE

Polyvinyl chloride (PVC) and other halogen-containing plastics have been produced and used around the world for at least 50 years. At the end of their intended life these materials can be recycled mechanically by moulding into new forms and structures, however there is a finite limit to the number of times that mechanical recycling can be employed before a more permanent recycling or disposal solution is needed. Chemical recycling is the process of converting waste plastics into their constituent chemical components, such that they can be used to make new plastics or other chemical products. Alternatively plastics can be disposed of to landfill, or used as a fuel source for power generation in waste-to-energy installations.

Both chemical recycling and power generation present significant challenges for PVC and other halogen-containing plastics. Both processes require the breaking of chemical bonds within the plastic, which results in depolymerisation to produce smaller molecules that can either be combusted or recovered as chemical products. Temperatures of 250-500 °C are typically required to break the bonds and achieve depolymerisation of PVC. Depolymerisation products are an acid-rich liquid stream that contains the majority of the chlorine from the original plastic, a hydrocarbon liquid fraction, a non-condensable gas fraction, and a residual solid.

Conventional processing technologies for recycling PVC require contacting granular or powdered plastic with a hot gaseous medium. Large particle sizes cannot be accommodated due to their inherent low thermal conductivity, and such large particles require the addition of a shredding or milling step in order to reduce the size to <10 mm. The hot gas has to (i) be at a higher temperature than the required temperature for depolymerisation in order that sufficient heat transfer can take place, typically 500-600 degrees C; (ii) have an oxygen concentration below the lower flammability limit for the combustible products, typically less than 2%. Heat is transferred from the gas to the plastic, providing the energy needed to heat the plastic and break the chemical bonds. Depolymerisation products are produced in vapour form, and are evolved into the hot inert gas within the reactor. The normal boiling point of the acid-rich products is around 100-120 degrees C, so they remain in vapour form within the reactor and carried with the remaining solid within the hot gaseous medium. Separation of the residual solid is carried out before cooling and condensing the products to recover the liquid components. The presence of large amounts of hot acid creates a very corrosive environment, which is very challenging for conventional coolers and condensers to handle in a robust manner. Acid products are also challenging to handle within the high temperature regions of the process during the primary depolymerisation.

The residual solid has a high carbon content, which derives from the carbon backbone of the PVC. It has a high surface area, contains components of the polymer that are not depolymerised, and also the remains of any inorganic additives from the original polymer such as pigments. The hot environment in which the depolymerisation takes place, coupled with the presence of solid residue acts to further break down the hydrocarbon product and causes side reactions that promote the formation of chlorinated hydrocarbons, thereby reducing the inherent value of the hydrocarbon product.

As a consequence, chemical recycling of PVC using conventional heating or combustion techniques is not currently carried out. Alternative technology solutions are required that overcome the inherent challenges of handling acidic products whilst maximising the quality of the produced hydrocarbon phase.

Microwave heating is one such method that can transfer energy to PVC whilst maintaining a cold surrounding environment during the depolymerisation process. Previous approaches have attempted to utilise this technology, but have found that levels of microwave absorption within PVC are relatively low and consequently required the use of additives that absorb the microwaves and subsequently transfer heat to the plastic by conventional means. U.S. Pat. No. 5,387,321 uses a carbonaceous material for this purpose. US20130165710 describe the use of a sensitizer, which in turn heats the plastic. WO200753088 details the use of a microwave-absorbing substance mixed within the feedstock. All of these innovations, and other similar approaches mean that the environment temperature surrounding the plastic is hot, and therefore these innovations do not enable the formation of high quality hydrocarbon product that results from a cold surrounding.

Alternative approaches to overcome the low degree of microwave absorption are the use of microwaves to generate a plasma, as described in US20190284476, or an electric arc, as described in CN111097350. In both cases energy is transferred to the plastic by the high temperature environment of the plasma or the electric arc, and not directly by the microwaves themselves. These approaches do not maintain a cold surrounding, and inhibit the quality of the products resulting from the depolymerisation process.

Other innovations have attempted to use microwave heating more directly, without the use of microwave-absorbing additives. JPH10185140 uses a microwave-heating moving bed, with a dispersion media added to improve the flow of plastic and degradation products through the reactor.

JPH0971683 utilises a mixed waste material with iron and copper-based microwave-absorbing species already present, and describes a process whereby the PVC-containing waste is dispersed in air during the treatment. This document cites the use of multiple microwave sources to generate even heating of the chlorine-containing waste. This gives rise to a widely distributed electric field within the reactor and a low power density. In this case, the power density is too low to induce direct pyrolysis of PVC without the presence of the microwave-absorbing additives that are inherent within the teaching of this application.

Processes that rely on moving beds or a dispersed solid within a gas have three major drawbacks: (1) They require precise control of the particle size in order for the process to function effectively, (2) The residence time of the plastic within the reactor cannot be precisely controlled, resulting in varying levels of energy absorption and variable product quality, (3) The residual solid can be entrained with the product vapours and result in the need for further separation stages within the system.

‘Wet’ processes have been attempted in several innovations. These processes utilise a chemical reaction to break the bonds and separate the Chlorine, rather than high temperatures. Glycolysis or acidolysis reactions are typically employed for this purpose, at temperatures up to 200 °C. CN102127246 reports a wet process that uses microwave heating to separate heteroatoms, and JP4605602 reports a glycolysis process for removal of halogen atoms from plastics. In both cases the chlorine products need to be separated from the reaction media, and there is no hydrocarbon product as is the case in thermal depolymerisation processes.

WO0228609 discusses the addition of a liquid to soften crumb from waste tyres prior to a shearing process to separate rubber from metal.

It is against this background that the disclosure has been devised.

### SUMMARY OF THE DISCLOSURE

In one aspect, there is provided a method of processing a feedstock, wherein the feedstock includes halogenated polymer particles, wherein the halogenated polymer particles have a polymer type that has a first pre-determined solubility parameter.

The method includes pre-treating the feedstock by applying a solvent to the feedstock, wherein the solvent has a second pre-determined solubility parameter, and wherein the second pre-determined solubility parameter substantially matches the first solubility parameter; and following pre-treating, irradiating the feedstock with microwave radiation using a microwave reactor.

In another aspect, there is provided a method of pre-treating the plastic feedstock to improve or enhance the ability of the plastic feedstock to directly absorb microwave radiation before the plastic feedstock undergoes microwave pyrolysis.

The method may include applying a solvent to the feedstock.

The solvent may be applied by any suitable means for example spraying, pouring, mixing, or otherwise ensuring contact between the feedstock and the solvent.

The solvent has a second pre-determined solubility parameter that substantially matches the solubility parameter of the plastic feedstock material (i.e. the first pre-determined solubility parameter). Preferably, the solvent may have a solubility parameter value within +/−1.0 MPa^{0.5} of the solubility parameter of the plastic feedstock. In some cases the solvent may have a solubility parameter value within +/−5.0 MPa^{0.5} of the solubility parameter of the plastic feedstock.

The solvent may include a swelling agent, which may be a liquid swelling agent. The solvent may include 5 weight percent of the total feedstock delivered to a microwave reactor for microwave pyrolysis. Preferably, the solvent applied to the feedstock includes between 0.1% and 20% per unit mass of the feedstock.

The plastic feedstock may include polyvinyl chloride (PVC). The feedstock may have a particle size above 10 mm. The feedstock may have a particle size below 20 mm. The feedstock may have a particle size below 50 mm. The feedstock may be delivered to the microwave reactor in a batch process or in a continuous process. The feedstock may be delivered to the microwave reactor by a conveying system.

There is further provided a method for recycling or depolymerising a plastic material using microwave pyrolysis. The method may include pre-treating a plastic feedstock as described in any of the above paragraphs.

The method may include delivering microwave radiation to the pre-treated plastic feedstock in a microwave reactor, i.e. energising a microwave reactor to irradiate the feedstock with microwave radiation. The method may include delivering microwave radiation to the pre-treated plastic feedstock in a microwave reactor in the absence of a dispersing media for absorbing and delivering microwave radiation to the plastic feedstock. Thus, the method may include directly heating the pre-treated plastic feedstock using microwave radiation.

The method may include delivering the pre-treated plastic feedstock to the microwave reactor via a conveying system. The conveying system may include an auger, a rigid conveyer or a segmented conveyor.

The feedstock may pass through an electromagnetic filter before entering the microwave reactor and/or after exiting the microwave reactor. The or each electromagnetic filter may provide around −60 dB of attenuation. The or each electromagnetic filter may provide at least 40 dB of attenuation, and preferably at least −60 dB of attenuation.

The microwave reactor may be controlled to generate a power density of between 10⁶-10⁸ W/m³, or more preferably between 10⁷-10⁸ W/m³.

The method may further include extracting gaseous products of depolymerisation of the feedstock. The gaseous and vapour products may be extracted with the use of a sweep gas flowed through the microwave reactor. The sweep gas may be inert.

The interior of the microwave reactor may be maintained below a temperature of 70 °C during the microwave pyrolysis process, and preferably below 50 °C. The temperature of the microwave reactor may be maintained by the sweep gas. Carrying out the depolymerisation process in a lower temperature environment overcomes numerous challenges: (1) The acidic product can be condensed directly when it is evolved into the cold surrounding environment, and recovered without the need to contact heat exchange surfaces that can be corroded, (2) The hydrocarbon product is evolved into a cold surrounding, which results in direct condensation and solidification and limits further degradation and side reactions that can form chlorinated hydrocarbons.

There is provided an apparatus for performing a microwave pyrolysis process on a plastic feedstock. The apparatus includes a microwave reactor for heating a plastic feedstock. The apparatus further includes a conditioning or pre-treatment section in which the plastic feedstock is pre-treated before being fed into the microwave reactor.

The plastic feedstock may be mixed with a solvent in the conditioning section. The solvent may have a solubility parameter that substantially matches the solubility parameter of the plastic feedstock material. Preferably, the solvent may have a solubility parameter value within +/−1.0 MPa^{0.5} of the solubility parameter of the plastic feedstock. In some cases the solvent may have a solubility parameter value within +/−5.0 MPa^{0.5} of the solubility parameter of the plastic feedstock.

The solvent may be selected from the group comprising: acetone, toluene, tetralin, dichloromethane, and methyl ethyl ketone.

Mixing the plastic feedstock with the solvent in the conditioning section enables subsequent effective direct heating of the plastic by the microwave field, and so allows for the cold surrounding environment around the plastic feedstock to be sustained. This approach directly overcomes limitations within the prior art that relies on the use of microwave-absorbing additives or dispersion of the feedstock within a fluidising medium to enable indirect heating of a feedstock using a microwave field.

There is further provided a method of processing a plastics feedstock, including providing a feedstock containing halogenated polymeric particles, delivering the feedstock to a microwave reactor, irradiating the feedstock with microwave energy in the reactor, and maintaining the temperature within the reactor below a predetermined temperature of 70 °C, and more preferably 50 °C. The method may include initiating pyrolysis of the feedstock by irradiating the feedstock with microwave energy in the microwave reactor. The method may include maintaining the temperature within the microwave reactor below a predetermined temperature of 70 °C, and more preferably 50 °C, during pyrolysis of the feedstock.

The method may include delivering a sweep gas to the microwave reactor at a temperature below the predetermined temperature to assist in maintaining the microwave reactor below the predetermined temperature.

The feedstock may have a particle size above 10 mm. The feedstock may have a particle size below 20 mm. The feedstock may have a particle size below 50 mm.

The feedstock may have a polymer type that has a first pre-determined solubility parameter;
wherein the method may further include, prior to irradiating the feedstock in the microwave reactor, pre-treating the feedstock by applying a solvent to the feedstock, wherein the solvent has a second pre-determined solubility parameter; wherein the second pre-determined solubility parameter substantially matches the first solubility parameter.

The second solubility parameter may match the first solubility parameter to within +/−5.0 MPa^{0.5}. The second solubility parameter may match the first solubility parameter to within +/−1.0 MPa^{0.5}.

The solvent applied to the feedstock may include greater than 0.1% per unit mass of the feedstock. The solvent applied to the feedstock may include less than 20% per unit mass of the feedstock.

The processing method may subject the plastic feedstock to a high intensity electric field that sustains an average power density of at least 10⁷ W/m³. The residence time of the feedstock within the high intensity electric field may be controlled precisely, and regulated between 10-50 seconds. The high intensity electric field and precise residence-time control minimise the degree of overheating of the PVC, and promote a cold-surrounding environment temperature of 50-70 °C.

An inert gas may be supplied at atmospheric temperature, and used to entrain the produced acidic products from the microwave pyrolysis process and directly condense and entrain the organic products.

The entrained products may be removed from the microwave reactor to a direct contact separator, which uses a circulating flow of cold water or weak hydrochloric acid solution to absorb the acid component of the product vapour and directly condense the organic product. The direct contact separator may employ a secondary organic liquid, such as kerosene, in addition to the aqueous fluid to promote absorption of the organic product and to mobilize high molecular weight wax products that would otherwise foul the internal components within the condenser.

Products from the direct contact condenser may be allowed to flow under gravity to a phase separator, where the organic product is withdrawn at the top along with the organic liquid used within the direct contact separator. The acidic product is removed at the base of the phase separator with the water or acid solution that is added to the direct contact separator.

In another aspect, the disclosure resides in a product produced by the method of any of the preceding paragraphs, and in particular in an organic product produced by the method of any of the preceding paragraphs.

### BRIEF DESCRIPTION OF THE DRAWINGS

In order that the disclosure may be more readily understood, preferred non-limiting embodiments thereof will now be described, by way of example only, with reference to the accompanying drawings, in which like features are assigned like reference numbers, and in which:

FIG. 1 is a process flow diagram showing exemplary apparatus according to the disclosure;

FIG. 2 is a process flow diagram showing an alternative configuration of apparatus according to the disclosure; and

FIG. 3 is a schematic showing an alternative configuration of apparatus according to the disclosure.

### DETAILED DESCRIPTION OF EMBODIMENTS OF THE DISCLOSURE

Examples of the disclosure include a dry process for the depolymerisation of PVC, using microwave heating to produce an acid-rich product and a liquid hydrocarbon product.

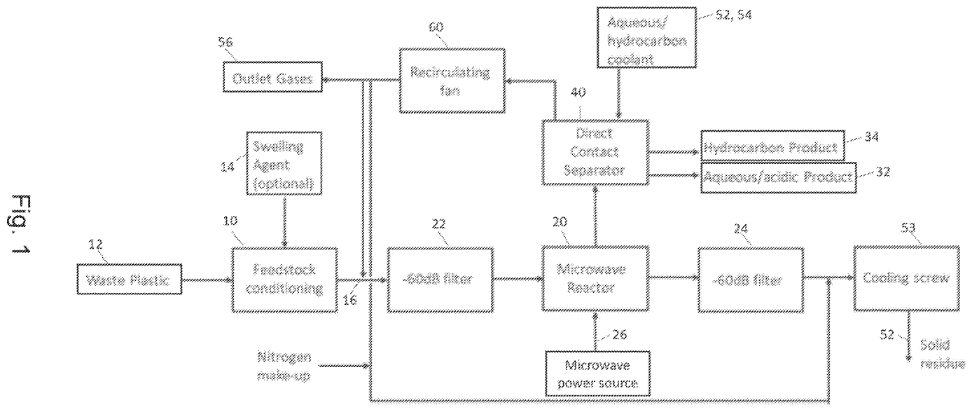

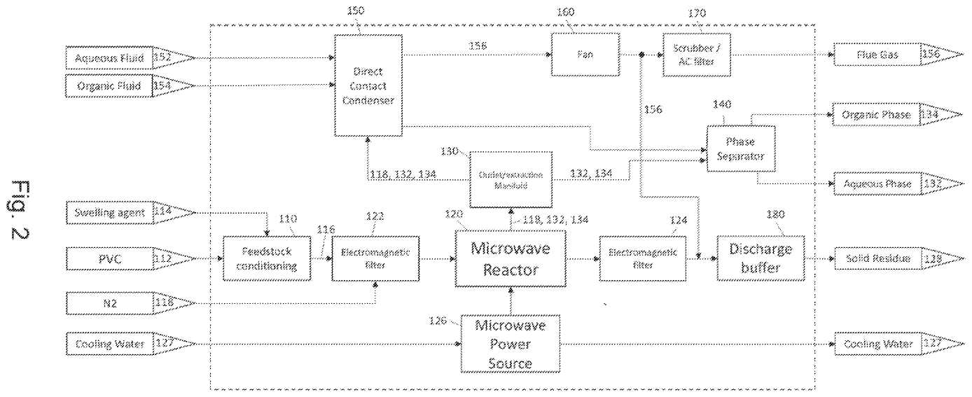

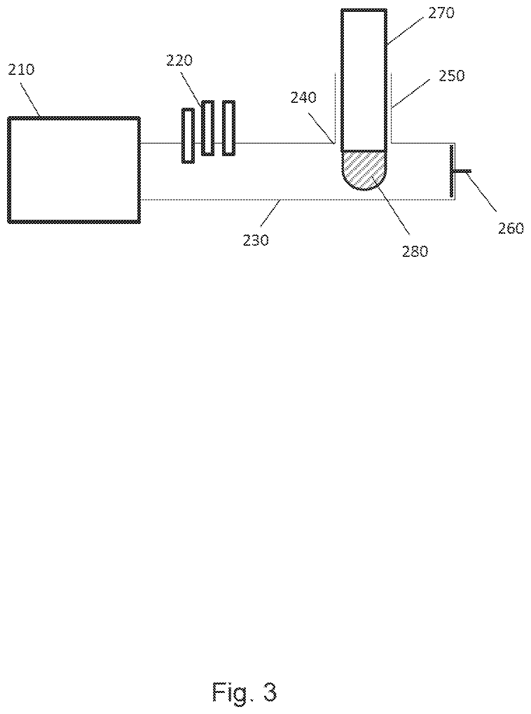

FIG. 1 and FIG. 2 are process flow diagrams which each show an embodiment of a process according to the disclosure, also referred to as a depolymerisation process. FIG. 3 also illustrates an embodiment of a process according to the disclosure, namely a setup, which may be a laboratory-scale setup, that allows batch depolymerisation of PVC with swelling-agent applied.

Turning first to FIG. 1, a depolymerisation process, which in this example is a PVC depolymerisation process, is illustrated.

According to FIG. 1, a process feedstock 12, which includes waste plastic such as PVC, is added to a feedstock conditioning section 10 along with a swelling agent 14. The conditioned feedstock 16 is conveyed through an electromagnetic filter 22 into a microwave reactor 20 where microwave heating of the conditioned feedstock 16 occurs. The microwave reactor 20 is supplied with power from a microwave power source 26.

Depolymerisation of the conditioned feedstock 16 produces hydrocarbon product 34, aqueous/acidic product 32, gaseous phase 56, and solid residue 52. The hydrocarbon product 34, aqueous/acidic product 32, and gaseous phase 56 pass through a direct contact separator 40, where the hydrocarbon product 34 and the aqueous/acidic product 32 are condensed and removed. Coolant in the form of aqueous fluid 52 and hydrocarbon fluid 54 is used in the direct contact separator 40 to condense the hydrocarbon product 34 and aqueous/acidic product 32.

Residual solid feedstock 52 that does not depolymerise within the microwave reactor 20 is conveyed out of the microwave reactor 20 through the second electromagnetic filter 24 that provides −60 bB of attenuation. The solid residue 52 is deposited into a cooling screw 53, where it cools before exiting the PVC depolymerisation process.

The gaseous phase 56 passes through a recirculating fan 60 before either re-entering the PVC depolymerisation process at either end of the microwave reactor 20 via the two electromagnetic filters 22, 24, or exiting the process as an outlet gas.

Turning now to FIG. 2, this illustrates a PVC depolymerisation process in accordance with another embodiment of the disclosure.

The process feedstock 112 includes waste plastic such as PVC, with particle sizes ranging from 1 mm to 50 mm, but typically with an average particle size of 20 mm. Sieve analysis using a column of sieves with a range of sieve sizes may be conducted on samples from each batch of waste plastic feedstock 112 to determine the particle size distribution of the waste plastic feedstock 112 which is to be processed. In this example, sieve analysis is undertaken using a standard procedure of: weighing a representative sample of the mixed waste plastic feedstock 112 to determine the total mass, placing the sample into the top sieve of a column of sieves with meshes of decreasing sieve size, shaking the column adequately, weighing the portion of sample retained on each sieve, and dividing the mass of sample per sieve by the total mass to give a percentage of the sample at each sieve size.

In some embodiments, a sieving step (not shown) is undertaken at the beginning of the process, prior to conditioning of the waste plastic feedstock 112, in order to control the particle size which is to be processed. The sieving step may include a suitably-sized aggregate screening system for sieving the waste plastic feedstock 112. Preferably, the largest mesh of the screening system has a sieve size of 50 mm (2 inches). In other embodiments, the largest mesh may include a different sieve size, e.g. 20 mm. By way of such a process, which is known in the art, a granular waste plastic feedstock 112 of a known maximum particle size may be obtained. Typically, waste plastic feedstock 112 is commercially available in granular form at defined sizes.

The process of the disclosure advantageously allows larger particle sizes of waste plastic feedstock 112 to be processed compared with conventional thermal recycling technologies which typically require particle sizes of less than 10 mm. Small particles are required when conventional heat transfer is the rate limiting step within a process, as is the case for conventional thermal processing technologies. With microwave heating of particles less than 100 mm, the energy transfer is instantaneous and there is no thermal conductivity limitation. As a result, there is no restriction on the particle sizes that can be heated due to thermal conductivity limitations, and the overall process system benefits from not requiring a size-reduction step to pre-condition the waste plastic feedstock 112. Larger particles also promote disengagement of solid and liquid/vapour products, and the process system benefits from not requiring intensive solid separation stages after the heating section.

Referring still to FIG. 2, the PVC depolymerisation process includes a feedstock conditioning section 110 where the waste plastic feedstock 112 is contacted with a swelling agent 114. The feedstock conditioning section 110 includes a feed hopper (not shown) in this embodiment, into which the waste plastic feedstock 112 is transferred in batches and then contacted with the swelling agent 114. It will be appreciated that, in other embodiments, other appropriate apparatus may be used for contacting the waste plastic feedstock 112 with the swelling agent 114. The skilled person understands that there are other appropriate equipment and methods for contacting liquids and solids.

For conditioning of the waste plastic feedstock 112, liquid swelling agents 114 having a Hildebrand solubility parameter value within +/−1.0 MPa^{0.5} of the Hildebrand solubility parameter of the plastic of the waste plastic feedstock 112 are optimum. However, the PVC depolymerisation process can operate effectively using liquid swelling agents 114 having Hildebrand solubility parameters that fall within +/−5.0 Mpa^{0.5} of the equivalent solubility parameter of the plastic of the waste plastic feedstock 112. This is because materials with similar Hildebrand solubility parameters are more likely to be miscible and cause swelling of the plastic.

PVC has a Hildebrand solubility parameter of 19.5 Mpa^{0.5}. For conditioning of the waste plastic feedstock 112 in the conditioning section 110, liquid swelling agents 114 in the form of, for example, ketones such as acetone (having a Hildebrand solubility parameter of 19.9 Mpa^{0.5}) or toluene (having a Hildebrand solubility parameter of 18.3 Pa^{0.5}) may be used. Dichloromethane (having a Hildebrand solubility parameter of 20.2 Mpa^{0.5}) and Methyl ethyl ketone (MEK) (having a Hildebrand solubility parameter of 19.3 Mpa^{0.5}) are other examples of liquid swelling agents 114 which may be used for PVC. The use of other suitable liquid swelling agents 114 in the conditioning section 110 is possible. For example, other aromatic hydrocarbons, tetralin, or the crude hydrocarbon product that is produced by the depolymerisation process may be used. The liquid swelling agents 114 used in the conditioning section 110 are preferably undiluted, at a concentration of 100%.

The liquid swelling agent 114 typically includes 5 wt % (dry weight) of the total waste plastic feedstock 112 added to the microwave reactor 120 (i.e., for every 1 kg of waste plastic feedstock 112, 50 g of liquid swelling agent 114 is added). Liquid swelling agent 114 dosages of <0.1 wt % do not typically result in improvement in microwave absorption behaviour, whilst concentrations >20 wt % offer no additional benefit and require more energy to process.

On contact with the waste plastic feedstock 112, the liquid swelling agent 114 diffuses into the plastic. The polymer chains of the waste plastic of the feedstock 112 are moved apart by the liquid swelling agent 114, expanding the polymer matrix. The use of the liquid swelling agent 114 therefore promotes chain mobility and enhances the effect of subsequent microwave heating of the waste plastic feedstock 112.

The liquid swelling agent 114 may be in contact with the plastic for between 15 seconds and 15 minutes before progressing through the next stage of the PVC depolymerisation process in order to sufficiently condition the waste plastic feedstock 112. Between one and two minutes of contact time between the waste plastic feedstock 112 and the liquid swelling agent 114 is generally sufficient before progressing through the process. Conditioning of the waste plastic feedstock 112 is carried out at room temperature in this example, however other temperatures may be employed.

Where known systems use microwave-susceptors to improve microwave heating behaviour, the process of the disclosure enables and improves direct interaction of microwave energy with the polymer chains that form the waste plastic of the feedstock 112. As explained above, the use of swelling agents to condition the feedstock addresses limited polymer chain mobility to enable effective microwave heating to take place.

Direct microwave heating enables a consistent temperature profile to be maintained within the plastic of the feedstock 112, and assists in avoiding hot-spots. Hot-spot formation occurs when there is a very large temperature gradient within the plastic, and this arises when microwave-absorbent and microwave-transparent materials are located adjacent to each other. Microwave-susceptors added to plastic cause hot-spot formation, and the subsequent high localised temperatures lead to poor control of the depolymerisation process and significant degradation of the primary depolymerisation products. Elimination of hot-spots allows for the quality of primary depolymerisation products to be maintained, and this is achieved through the disclosure by dissipating the microwave energy throughout the entire volume of the plastic rather than in a finite number of microwave-absorbent zones. That is, the use of swelling agents to condition the plastic feedstock 112 before microwave heating eliminates hot-spots by promoting microwave absorption within the entire volume of the plastic.

Still referring to FIG. 2, once the waste plastic feedstock has undergone conditioning in the conditioning section 110, the conditioned waste plastic feedstock 116 (hereafter referred to as the conditioned feedstock 116) is transferred through a feed hopper (not shown) to a conveying system (not shown), which conveys the conditioned feedstock 116 into a microwave reactor feed system (not shown). The conveying system can take the form of an auger, a rigid conveyor or a segmented conveyor. The conveying system may be an off-the-shelf component, with numerous options available as detailed within Beneroso et al., Chemical Engineering Journal 316 (207) 481-498, “Microwave pyrolysis of biomass for bio-oil production: Scalable processing concepts”. Referring to Beneroso et al., conveying options that are suitable for processing PVC include linear conveyors, rigid rotary conveyors and auger-based processes. The rigid linear conveyor system detailed within WO2017178793A1 is another example of a suitable conveying system, and WO2015110797A3 details an alternative configuration for a suitable auger-based conveying system.

Before entering the microwave reactor 120, the conveyor and conditioned feedstock 116 pass through an electromagnetic filter 122 that provides −60 dB of microwave attenuation. The electromagnetic filter 122 is designed using established electromagnetic principles and based on the geometry of the microwave reactor 120 and the conveyor. Another −60 dB electromagnetic filter 124 is located on the exit side of the microwave reactor 120 for the solid stream which exits the microwave reactor 120. The electromagnetic filters 122, 124 act as entry and exit gates for the microwave reactor 120, and thereby allow entry and exit of the conditioned feedstock 116 whilst guarding against microwave leakage.

The microwave reactor feed system can handle particle sizes ranging from 1 mm to 50 mm. The microwave reactor feed system transfers the conditioned feedstock 116 into the reactor section of the microwave reactor 120.

Depolymerisation of the conditioned feedstock 116 takes place in the reactor section of the microwave reactor 120. Microwave heating occurs directly and in the presence of an inert gas that is at a temperature below 50 °C, thus creating a cold surrounding environment.

The reactor section of the microwave reactor 120 includes a microwave power source 126 for a microwave that supports a high intensity electric field, such that the power density within the heated phase is typically above 10⁷ W/m³, but could range from 10⁶-10⁸ W/m³. The electric field intensity within the microwave reactor 120 is controlled to heat the plastic evenly. The electric field intensity within the microwave reactor 120 is controlled to achieve a microwave power density that favours the formation of high value organic products and minimises the side reactions that produce chlorinated hydrocarbons. Power density (power/volume) values in excess of 10⁷ W/m³ are required to minimise side reactions and secondary decomposition, and to minimise heat losses to the reactor that act to include the cold surrounding environment.

The microwave power source 126 has a cooling water supply 127 within the range of 18-25 °C that removes excess heat from the transformer and reflected power.

The PVC depolymerisation process within the microwave reactor 120 is continuous, operating at 1-10 kg/h of conditioned feedstock 116 when using a microwave frequency of 2450+/−20 MHz, and 50-2000 kg/h of conditioned feedstock 116 when operating at 905+/−20 MHz.

The operating frequency of the microwave reactor 120 is 885-925 MHz in this embodiment, but may differ in other embodiments. For example, in some embodiments the operating frequency may be 2430-2470 MHz or 420-450 MHz. The reactor of the microwave reactor 120 may be sized according to the principles detailed within Robinson et al., Chemical Engineering Research and Design. 88 (2010) 46-154, “Scale-up and design of a continuous microwave treatment system for the processing of oil-contaminated drill cuttings”, and according to the power density range specified above. Specifically, the iterative procedure summarised on p149 of Robinson et al. shows the steps that may be followed to achieve both high and uniform power density within the reactor, and FIGS. 8, 9, 10 & 14 show examples of the electric field and power density distribution that may be achieved as a result.

The residence time within the microwave reactor 120 is controlled by regulating the speed of the conveyor, and is typically 10-50 seconds. The absorbed energy is regulated by varying the depth of conditioned feedstock 116 on the conveyor and the power applied for a given conveying speed. Energy inputs >3.5 kJ/g are required when liquid swelling agent concentration exceeds 20%. If liquid swelling agent 114 is not used, energy inputs from 2-3 kJ/g are required to depolymerise all halogen-containing plastics in the waste plastic feedstock 112.

The amount of energy absorbed by the conditioned feedstock 116 may be regulated by changing any one of multiple factors, including but not limited to, power input, flowrate of conditioned feedstock 116, volume through which power is absorbed, configuration of conveyance system and cross sectional geometry of the microwave reactor 120.

The speed of the conveying device regulates the residence time of the conditioned feedstock 116 within the microwave reactor 120. The conditioned feedstock 116 cannot spend longer, or less time in the reactor than the conveyor allows. This is in contrast to a fluidised bed or stirred tank reactor where there is an inherent residence time distribution. With a precisely-controlled residence time, it is possible to regulate the exact amount of energy to the conditioned feedstock 116 to achieve depolymerisation whilst minimizing side reactions. The energy delivered to the plastic of the conditioned feedstock 116 is a function of the microwave power and the flowrate of plastic within the microwave reactor 120.

Inert sweep gas 118 is fed into the microwave reactor 120, with injection points positioned at the inlet to the electromagnetic filter 122. The gas 118 temperature is typically <30 °C when it is injected into the microwave reactor 120, and this enables the ability to maintain a temperature of <50 °C within the headspace of the reaction zone region of the microwave reactor 120, i.e., a low temperature environment. Nitrogen is used as the sweep gas in this embodiment, but other inert gases such as CO₂ and argon may be used, alone or in combination, in other embodiments.

The PVC depolymerisation process operates at atmospheric pressure in the embodiment, but in other embodiments sub-atmospheric pressures (namely high-vacuum environments) may be acceptable.

Products from depolymerisation (in the form of acidic products 132 and hydrocarbon (organic) products 134) are directly condensed in the low temperature environment within the microwave reactor 120, and are entrained within the inert sweep gas 118 and removed via extraction manifolds 130. The extraction manifolds 130 are designed to allow some deposition of the condensed acidic products 132 and hydrocarbon products 134 to occur, with this condensate diverted to a phase separator vessel 140 rather than flowing back into the microwave reactor 120.

Downstream of the manifold 130, further acidic products 132 and hydrocarbon products 134 are cooled and condensed in a direct contact condenser 150, where they then are directed to pass through the phase separator 140. Two contact fluids are typically used within the condenser 150 as the cooling medium; water or a water/acid mixture (aqueous fluid) 152 to promote removal of the acidic product 132 from PVC pyrolysis, and an organic solvent (organic fluid) 154 to promote removal of the organic pyrolysis (hydrocarbon) product 134. Recovered hydrocarbon product 134 and/or acidic product 132 can be used as the cooling medium for the direct contact condenser 150. Freshly-supplied make-up aqueous and/or organic fluids may also be used as the cooling medium.

The acid product is formed in the same manner as in conventional thermal technologies. Conventional technologies produce acids in vapour form and at temperatures of the order of 500 degrees C. The acids are highly corrosive in this state, and subsequent cooling and condensation is very challenging due to the need for metallic heat transfer surfaces.

In the process of the disclosure, the acids are condensed within the sweep gas as the reactor environment temperature is maintained, for example, below 50 degrees C, whilst the boiling point of the acid product is in excess of 100 degrees C. The acid forms small droplets upon condensation, which are entrained with the sweep gas and carried away from the reactor. The low temperature of the acid-containing sweep gas means that materials of construction can be employed to handle the product that cannot be used with conventional technologies. By virtue of the disclosure, a direct contact cooling vessel can be situated immediately downstream of the reactor, and be made from glass-reinforced plastic, or other polymer-lined vessel that is resistant to the acid product. The cold surrounding enabled by the disclosure therefore allows the acid to be handled safely, in a manner that is not possible with conventional techniques.

The organic product is formed in the same manner as it does in conventional thermal technologies. Conventional technologies produce organic products in vapour form and at temperatures of the order of 500 degrees C. The organic products are prone to secondary and tertiary degradation reactions under these conditions, and can form undesirable by-products such as chlorinated hydrocarbons, benzene and carbon-rich char. In the current disclosure the organic products are produced at their reaction temperature, and are condensed and solidified within the cold surrounding and entrained within the sweep gas. The low temperature environment and rapid condensation of the organic products prevents secondary reactions, preserving the primary chemistry of the hydrocarbon product from the pyrolysis process. The organic product that arises from the process of the disclosure may be rich in naphthalene, which when purified is a stable solid product at room temperature. Naphthalene can be functionalised in a subsequent reaction step, for example to produce naphthalene epoxides or naphthoquinones to provide a platform for the production of dyes, insecticides and pharmaceutical products.

Liquid products from the direct contact condenser 150 flow under gravity to the phase separator 140, where the hydrocarbon products 134, organic fluid 154, and recovered swelling agent 114 are removed from the top of the separator 140 via a weir (not shown). Aqueous/acidic products 132, and aqueous fluid 152, are removed from the bottom of the separator 140 via a standpipe (not shown).

Non-condensable gases 156 are passed through a demister (not shown) before entering a recirculation system via the suction side of a recirculating fan 160, which pushes gases 156 back into the process section. FIG. 2 shows the gases 156 being recirculated into the conveying system after the second electromagnetic filter 124. However, it should be noted that gases 156 may be recirculated into both ends of the conveying system, prior to the first electromagnetic filter 122 and after the second electromagnetic filter 124. Gases 156 may also be only recirculated into the conveying system prior to the first electromagnetic filter 122.

Gases 156 are used to sustain both an inert atmosphere and a cold surrounding within the microwave reactor 120, whilst acting as an entraining medium to remove condensed droplets from the microwave reactor 120. The recirculation system includes a bleed point where gases 156 can be diverted to a scrubber 170, flare, or used as combustion fuel. Make-up of the recirculating gas 156 can be achieved using compressed nitrogen from a cylinder bank (not shown).

Residual solid feedstock 128 that does not depolymerise within the microwave reactor 120 is conveyed out of the microwave reactor 120 through the second electromagnetic filter 124 that provides −60 bB of attenuation. The solid residue 128 passes through a discharge buffer 180, where it can then be deposited into a cooling screw (not shown), where it cools before exiting the PVC depolymerisation process.

While FIGS. 1 and 2 show a continuous process for depolymerisation of PVC, FIG. 3 shows a batch microwave process for depolymerisation of PVC that has been combined with a suitable swelling agent, i.e. ‘swollen PVC’. Suitable swelling agents are those as described previously with respect to FIG. 2.

The system includes a microwave generator 210 operating at a frequency of 2.45 GHz and delivering an output power up to 6 kW. A 3-stub tuner 220 is used to tune the process and maximise the forward power absorbed by the swollen PVC 280 through impedance matching. Microwaves are directed using a waveguide 230, preferably a rectangular waveguide in the form of a WR340 waveguide or a WR430 waveguide, to a single mode microwave cavity 240. A chamber 270 that may, for example, take the form of a quartz reaction tube is located in the cavity 240 and contains the pre-swollen PVC 280. A short-circuit tuner 260 is used to position the swollen PVC 280 within the peak electric field region within the single mode microwave cavity 240. The reaction tube 270 is contained within an electromagnetic filter/choke 250 to contain the microwave field. An inert environment is maintained by purging nitrogen into the system. The depolymerisation products from the chamber 270 may be condensed and recovered using known condensation equipment.

As with the processes of FIGS. 1 and 2, the apparatus detailed in FIG. 3 allows swollen PVC 280 to be heated directly using microwaves, without the need for commonly-used microwave-absorbing additives such as carbon, metal oxides or silicon carbide.

Additional advantages of the disclosure include the following.

The ability to accommodate a large particle size range of feedstock. The conveying system can process any shredded material, and the particle size is not important for the function of the process nor the conveying method. The microwave depolymerisation process uses an electric field to provide direct heating of the plastic at a molecular level, and is not dependent on thermal conductivity. Unlike conventional heating, the microwave heating process does not require a large surface area for heat transfer and thus does not require small particle sizes in the manner that conventional processes do. The microwave depolymerisation process enables the use of large particle sizes, thus eliminating the need for feedstock size reduction. Separation of the solid char from the process products is less intensive when large particle sizes are used because the solids are not entrained with the gas flow as they are with conventional thermal depolymerisation processes. Consequently, the microwave depolymerisation process enables a simpler and less intensive separation process for the solid residue, which in combination with eliminating the size-reduction step means that there are fewer main plant items needed for the microwave depolymerisation process, which benefits the process footprint, capital cost and operating cost.

Direct condensation is promoted within the process, with condensate removed at the reactor outlet manifold and the direct contact condenser. This prevents condensate from re-entering reactor and removes the need for trace-heating the reactor and manifold walls. Trace-heating acts against sustaining a cold-surrounding environment, and the current disclosure overcomes this limitation.

No additional dispersing media are required in the described process. which allows for larger-sized feedstocks to be employed and further promotes the cold surrounding environment.

Hot-gas and solid contacting is not needed with this disclosure. Conventional heating technologies that are based on fluidised or spouted bed systems require a hot inert gas that is provided at a velocity that is sufficient to fluidise or agitate the plastic, with the gas/solid contact being a key feature of the process. The current disclosure does not require any gas/solid contact, with the role of the gas being to condense and entrain the evolved products rather than to interact with the plastic itself.

Possible variants to the above described process could reside in the following aspects:
Conveying method
Condensation/liquid separation system
Fluids used for direct condensation
Control system
Electromagnetic filter characteristic
Residue cooling technique

1-34. (canceled)

35. A method of processing a feedstock, wherein the feedstock includes halogenated polymer particles, wherein the halogenated polymer particles have a polymer type that has a first pre-determined solubility parameter, the method comprising;
pre-treating the feedstock by applying a solvent to the feedstock, wherein the solvent has a second pre-determined solubility parameter, and wherein the second pre-determined solubility parameter substantially matches the first solubility parameter; and
following pre-treating, irradiating the feedstock with microwave radiation using a microwave reactor.

36. The method of claim 35, wherein the second solubility parameter matches the first solubility parameter to within +/−5.0 MPa^{0.5}.

37. The method of claim 35, wherein the second solubility parameter matches the first solubility parameter to within +/−1.0 Mpa^{0.5}.

38. The method of claim 35, wherein the solvent applied to the feedstock includes greater than 0.1% per unit mass of the feedstock.

39. The method of claim 35, wherein the solvent applied to the feedstock includes less than 20% per unit mass of the feedstock.

40. The method of claim 35, wherein the solvent is selected from the group consisting of: acetone, toluene, tetralin, dichloromethane, and methyl ethyl ketone.

41. The method of claim 35, wherein the feedstock includes polyvinyl chloride.

42. The method of claim 35, wherein the feedstock has a particle size above 10 mm.

43. The method of claim 42, wherein the feedstock has a particle size below 20 mm.

44. The method of claim 35, wherein the feedstock is delivered to the microwave reactor in a batch process.

45. The method of claim 35, wherein the feedstock is delivered to the microwave reactor in a continuous process.

46. The method of claim 35, wherein the feedstock is delivered to the microwave reactor through an electromagnetic filter arrangement and/or wherein the feedstock is transported out of the microwave reactor through an electromagnetic filter arrangement.

47. The method of claim 35, wherein the feedstock is pre-treated in the absence of a dispersing media for absorbing microwave radiation.

48. The method of claim 35, wherein the microwave reactor is controlled to generate a power density of between 10⁶-10⁸ W/m³.

49. The method of claim 35, further comprising extracting gaseous and vapor products of depolymerization of the feedstock with the use of a sweep gas flowed through the microwave reactor.

50. The method of claim 53, wherein the sweep gas is inert.

51. The method of claim 35, further comprising maintaining the microwave reactor interior below a temperature of 70 °C whilst the feedstock is being irradiated, wherein the temperature of the microwave reactor is maintained by the sweep gas.

52. A method of processing a plastics feedstock, comprising:
providing a feedstock containing halogenated polymeric particles;
delivering the feedstock to a microwave reactor;
initiating pyrolysis of the feedstock by irradiating the feedstock with microwave energy in the microwave reactor; and
maintaining the temperature within the microwave reactor below a predetermined temperature of 70 °C during pyrolysis of the feedstock.

53. An organic product produced by the method of claim 35.

54. The method of claim 35, wherein the solvent is a swelling agent.
